(19) United States
(12) Patent Application Publication
(10) Pub. No.: US 2007/0282318 A1
(43) Pub. Date: Dec. 6, 2007

(54) Subcutaneous thermolipolysis using radiofrequency energy

(76) Inventors: Gregory J.R. Spooner, Kensington, CA (US); Scott A. Davenport, Half Moon Bay, CA (US); Allison Ferro, Fremont, CA (US); Dean A. MacFarland, Magnolia, MA (US); Kevin P. Connors, Sunnyvale, CA (US); Steven Christensen, Fremont, CA (US)

Correspondence Address: STALLMAN & POLLOCK LLP, 353 SACRAMENTO STREET , SUITE 2200, SAN FRANCISCO, CA, 94111, US

(21) Appl. No.: 11/804,002

(22) Filed: May 16, 2007

Related U.S. Application Data
(60) Provisional application No. 60/800,716, filed on May 16, 2006.
(60) Provisional application No. 60/900,820, filed on Feb. 12, 2007.

(51) Int. Cl. A61F 2/00; A61B 18/04

(52) U.S. Cl. 606/32; 607/101

(57) ABSTRACT

Disclosed herein are systems and methods that reduce, remove, shape, and/or sculpt sub-dermal fat layers by selectively heating fat tissue, or that reduce the appearance of cellulite, using low frequency RF energy applied through one or more skin contacting electrode carried on a handpiece. The handpiece is manipulated manually or automatically to continuously move the electrode(s) across the skin surface during RF delivery. A motion detector may be employed to determine the speed and/or direction of movement of the electrode, and operating parameters such as the amount of applied RF power may be modulated in response to feedback from the motion detector. One or more cooling modalities including thermoelectric cooling, and/or forced air cooling may be used to cool or minimize heating of the skin.

### CLAIM PRIORITY

This application claims the benefit of U.S. Provisional Application No. 60/800,716, filed May 16, 2006, and U.S. Provisional Application No. 60/900,820, filed Feb. 12, 2007.

### FIELD OF THE INVENTION

The present invention relates generally to treatment of body tissue, and more specifically to tissue treatment systems and methods using transcutaneous application of radiofrequency energy.

### BACKGROUND OF THE INVENTION

Adipose tissue is found in subcutaneous tissue throughout the human body. Adipose tissue, or fat, is formed of cells containing stored lipid. The fat is divided into small lobules by connective tissue septae.

Cellulite is a well known skin condition commonly found on the thighs, hips and buttocks. Cellulite has the effect of producing a dimpled appearance on the surface of the skin.

In the human body, subcutaneous fat is contained beneath the skin by a network of tissue called the fibrous septae. When irregularities are present in the structure of the fibrous septae, lobules of fat can protrude into the dermis between anchor points of the septae, creating the appearance of cellulite.

There is a large demand for treatments that will reduce adipose tissue volume, reshape the adipose tissue, and/or reduce the appearance of cellulite for cosmetic purposes. Currently practiced interventions for reduction/reshaping of adipose tissue include lipsosuction and lipoplasty, massage, low level laser therapy, external topicals, creams and preparations such as “cosmeceuticals.” Lipsosuction and lipoplasty are effective surgical techniques through which subcutaneous fat is cut or suctioned from the body. These procedures may be supplemented by the application of ultrasonic energy to emulsify the fat prior to its removal. Although they effectively remove subcutaneous fat, the invasive nature of these procedures presents the inherent risks of surgery as well as excessive bleeding, trauma, and extended recovery times.

Non-invasive interventions for subcutaneous fat reduction or diminution of the appearance of cellulite, including massage and low-level laser therapy are significantly less effective than the surgical interventions.

An ongoing need therefore exists for an effective modality by which subcutaneous fat tissue may be non-invasively reshaped, sculpted, and/or reduced for cosmetic improvement.

Some cosmetic skin treatments effect localized dermal heating by applying radiofrequency energy to the skin using surface electrodes. The local heating is intended to tighten the skin by producing thermal injury that changes the ultrastructure of collagen in the dermis, and/or results in a biological response that changes the dermal mechanical properties. The literature has reported some atrophy of subdermal fat layers as a complication to skin tightening procedures.

### BRIEF DESCRIPTION OF THE DRAWINGS

FIG. 1 is a simplified block diagram showing an exemplary RF thermolipolosis system.

FIGS. 2A and 2B are a plan view and a side elevation view, respectively, schematically illustrating edge effects along the perimeter of a circular electrode.

FIG. 2C schematically illustrates an exemplary range of movement of a circular electrode to minimize edge effect heating.

FIG. 2D schematically illustrates an exemplary axis of rotation for a circular electrode to minimize edge heating effects.

FIGS. 3A-3D illustrate a handpiece for the system of FIG. 1 in which, FIG. 3A is a perspective view, FIG. 3B is a bottom perspective view, and FIGS. 3C and 3D are cross-section views.

FIG. 4A is a front plan view showing an alternative embodiment of an energy applicator.

FIG. 4B schematically illustrates movement of the energy applicator of FIG. 4A within a target tissue region.

FIGS. 5A-5C are side elevation view showing three alternatives to the FIG. 4A embodiment, in which the electrode is electrically isolated from the thermoelectric cooler.

FIG. 6 is a side elevation view of an alternative to the FIG. 4A applicator which incorporates a system for moving the electrode over the tissue surface.

FIG. 7 is a top plan view schematically illustrating an electrode movement pattern for the FIG. 6 embodiment.

FIG. 8 is an alternative to the FIG. 4A applicator using an automated system for oscillating the electrode over the tissue surface.

FIG. 9 is a side view of an alternative to the FIG. 4A applicator in which real time RF power application may be modified as a functiontion of the direction and/or speed of movement of the electrode over the skin surface.

FIG. 10 schematically illustrates a speed and direction vector of the typed used to modulate an RF output power in the FIG. 9 embodiment.

FIG. 11 is a schematic side elevation view illustrating an energy applicator which uses an ohmic electrode arrangement. The electrode is shown in contact with skin.

FIG. 12 is a side elevation view similar to FIG. 11 showing an energy applicator having a capacitive electrode configuration.

FIG. 13 is a side elevation view similar to FIG. 11 showing an energy applicator having a resistive/dissipative electrode arrangement.

FIGS. 14-16 are schematic views similar to FIG. 13 showing energy applicators using alternative resistive/dissipative electrode configurations.

### DETAILED DESCRIPTION OF THE DRAWINGS

This application describes systems and methods that reduce, remove, shape, and/or sculpt sub-dermal fat layers by selectively heating fat tissue, or that reduce the appearance of cellulite, using low frequency RF energy applied through skin contacting electrodes. These systems and methods take advantage of the significant differences between the electrical and thermal properties of fat tissue compared with those of the surrounding tissues. For example, because fat tissue has significantly different values of permittivity and conductivity than skin, fascia and muscle, Joule heating using certain RF parameters occurs in the fat at a greater rate than in the surrounding tissues when the density of the applied electric field is substantially uniform. Additionally, fat possesses thermal properties (generally one-half the thermal conductivity of skin and less thermal capacity than skin) that permit fat tissue temperature to rise higher and at an accelerated rate relative to the skin and other tissues when exposed to selective heating of any type. Treatment parameters may thus be selected that will heat the subcutaneous fat, with minimal collateral heating of the skin, fascia and muscle.

By optimizing and controlling this selective fat heating while protecting the surrounding tissue from thermal damage, the disclosed system and method may be used to reduce, remove, shape and/or sculpt fat layers, adipose tissue, subdermal fat and/or to treat cellulite. Such changes to the fat tissue may result in smoothing or contouring of cosmetically undesirable body shapes, cellulite, facial shapes, and/or facial laxity. In this way, the disclosed system and method presents an alternative to invasive surgical procedures used for cosmetic purposes.

A number of parameters play a role in the disclosed systems for optimizing the effect of RF heating while minimizing collateral tissue damage. These parameters include electrode geometric dimensions, cooling modality (e.g. conduction, forced air, spray cryogen), the use and rate of electrode movement during treatment, and treatment area dimensions. The embodiments described below provide examples of certain combinations of combine these parameters, although other equally beneficial combinations may also be used and are contemplated within the scope of the present disclosure.

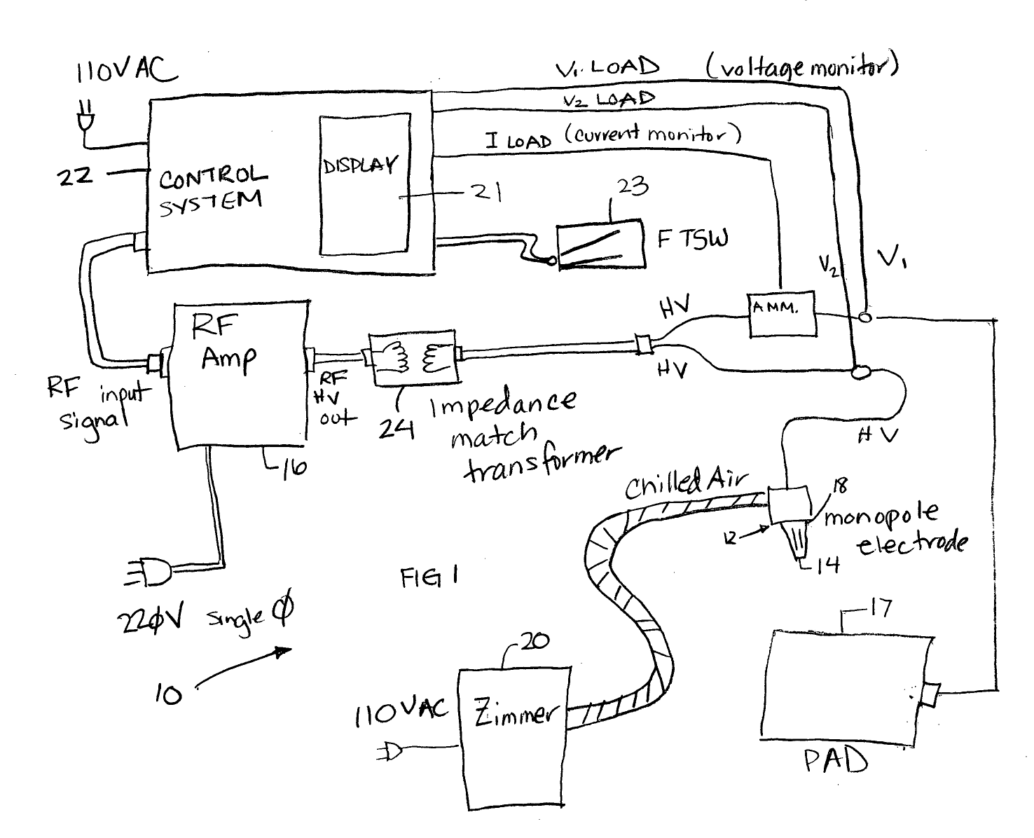

Referring to FIG. 1, general features of the system 10 include an applicator handpiece 12 including a monopolar skin-contacting treatment electrode 14 coupled to an RF power supply. Electrode 14 is preferably formed of a material such as copper that is both electrically and thermally conductive so as to permit cooling of the electrode during use of the system. In some embodiments, the electrode may be a single use product.

A dispersive electrode, such as large surface area pad 17 of the type well known in the art, is positionable in contact with the patient at a location remote from the treatment electrode 14 to provide a return path to the RF power supply.

A control system 22 is provided for controlling operation of the system 10. Control system includes a display 21 and one or more input devices such as touch screen features on the display allowing the user to select parameters for use of the system, and a footswitch 23 allowing the user to initiate delivery of RF energy to the treatment site.

In one example of an RF power supply configuration, control system 22 supplies a low voltage RF input signal to an RF amplifier 16, which generates a high voltage RF output. The output of the amplifier is connected to the electrode through an impedance matching transformer 24. A preferred transformer matches the output impedance of the RF power supply to that of a load, corresponding to the expected impedance of the electrode in contact with skin.

Voltage and current monitoring circuitry monitor the voltage and current applied to the electrode 14, and thus allow the control system 22 to determine the actual power supplied to the handpiece. The system may include impedance detection circuitry positioned to detect the impedance of the electrode in contact with tissue, and to provide feedback representing the measured impedance to the control system 22. For safety purposes, it is preferred that the control system 22 continuously track measured impedance of the applied RF power circuit. A rise in the local impedance of the treated tissue can indicate the presence of subcutaneous thermal effects, dermal bums, or poor electrode-tissue contact and will thus trigger an RF power shut-down.

Applicator handpiece 12 preferably includes one or more cooling elements 18 which employ one or more cooling modalities to cool tissue in treatment region. Many different types of cooling methods, such as cold air impingement, cryogen spray, or contact cooling systems (e.g. conduction cooling using an electrode cooled by a thermoelectric cooler) may be used to cool the tissue in the region undergoing treatment.

Depending on the type of cooling to be carried out, a cooling system 20 may be included. If conduction cooling using a thermoelectric cooler is employed, cooling can be achieved by simply energizing the thermoelectric cooler in the handpiece, thus obviating the need for a separate cooling system. If forced air cooling is to be used, cooling system 20 employs forced air cooling methods of the type achieved using the Cryo 5 cold air system manufactured by Zimmer MedizinSystems of Irvine, Calif. System 20 draws in air from the surrounding environment, chills the air and directs the cold air through a flexible hose 26 into the handpiece. In this embodiment, the cooling element 18 takes the form of one or more outlets in the handpiece for directing the chilled air onto the skin. Alternate cooling systems might direct cryogen to the handpiece for spraying onto the tissue, or circulate chilled water through the handpiece for conduction cooling. Control system 22 preferably controls the cooling system 20, although the cooling system 20 may instead operate independently of the control system 22.

Turning now to a discussion of electrode selection, treatment electrode 14 is preferably a monopolar surface contacting electrode. Bipolar electrode configurations might alternatively be used; however electric fields produced by a bipolar electrode array generally reach only shallower tissue regions such as the dermis. Heating of the subcutaneous fat and/or other subcutaneous tissue structures can be more readily achieved using a monopolar electrode, which generates electric fields that extend more deeply into the tissue.

In preferred electrode designs, the electrode lateral dimensions (e.g. the length and width of the electrode area contacting the tissue, or the diameter of a circular electrode) are selected to be large relative to the depth at which heating is desired. Whereas small area electrodes would produce an electric field distribution that rapidly diverges and heats only superficial skin layers, a large surface area electrode can more readily deliver an electric field to depths more suitable for fat tissue heating.

More specifically, the lateral dimensions of the contacting treatment electrode are preferably greater than:
sqrt[e]*d
where e is the real part of the complex dielectric constant at the applied frequency, and d is the target penetration depth, which corresponds roughly to the desired depth of heating. If the applied RF frequency is to be in the range of 10⁷ Hz, a value of e=20 is used in the equation based on typical e values for fat in the 10⁷ Hz frequency range (ignoring for the purpose of this example the skin/fat layer structure found in human tissue). In most instances, 0.3 cm<d<1 cm, and for the purposes of this example may be assumed to be 0.5 cm. According to this example, the lateral electrode dimensions will preferably exceed:
sqrt(20)*0.5 cm=2 cm.
Control of Edge Effect Heating

In selecting parameters for the system 10, it should be considered that when RF energy is applied to tissue via contact electrodes, the current density tends to be concentrated at the electrode edges. This effect, known in the art as the “edge effect,” results in higher current densities along the perimeter of contact electrodes than is found towards the center, with peak current densities appearing along sharp edges such as corners.

Because tissue heating increases with RF field concentration, the edge effect can cause tissue underlying the electrode edges and corners to experience higher temperatures than tissue underlying more central portions of the electrode. Embodiments described in this disclosure are configured to control or offset edge effect heating using various combinations of features such as (a) electrode movement features, (b) cooling features, and/or (c) electrode construction features. The discussion that follows will focus primarily on these three parameters. However, as discussed previously, selection of other features such as the electrode area to treatment area ratio and RF power levels also plays a role in minimizing edge effect heating.

Electrode Movement

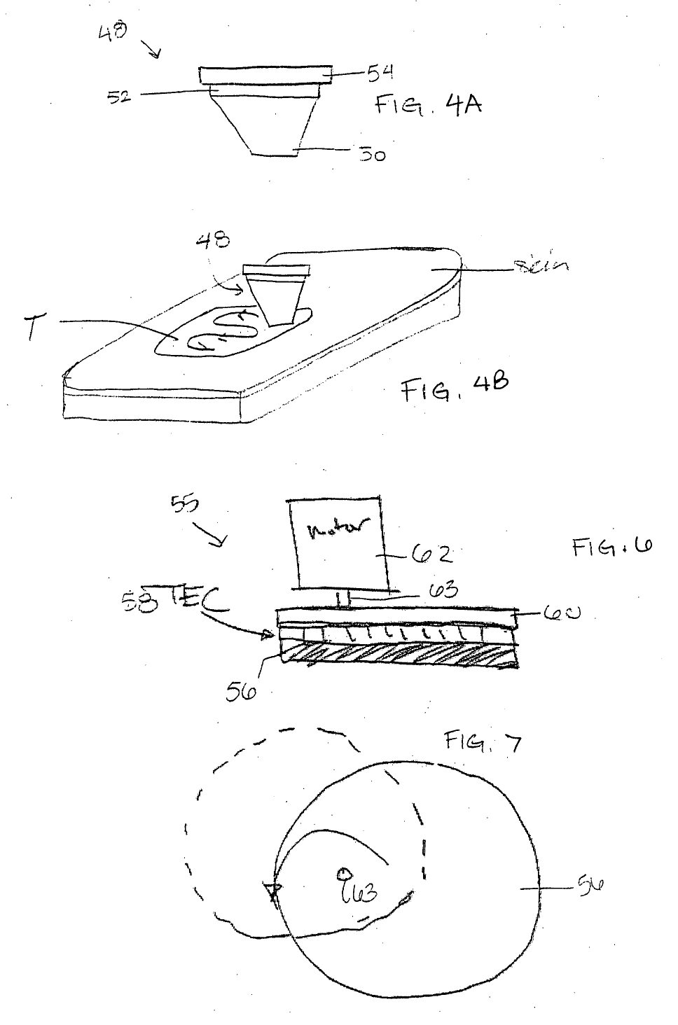

Keeping the handpiece 12 moving along the surface of the skin is useful for decreasing the edge effects of the electrode, and more evenly distributing the thermal effects of the treatment energy, thus reducing the chance that structural differences in the fat layer will lead to hot zones in some areas of the tissue and cooler zones in other areas. In some embodiments, the more even distribution of heat produced by moving the electrode can obviate the need for a cooling system. The rate at which the handpiece is moved across the skins can vary from a few millimeters (e.g. 1 or more) per second to several centimeters (e.g. 1-20) per second. As will be discussed in connection with the FIG. 4A-9 embodiments, electrode movement may be manual or semi-automated.

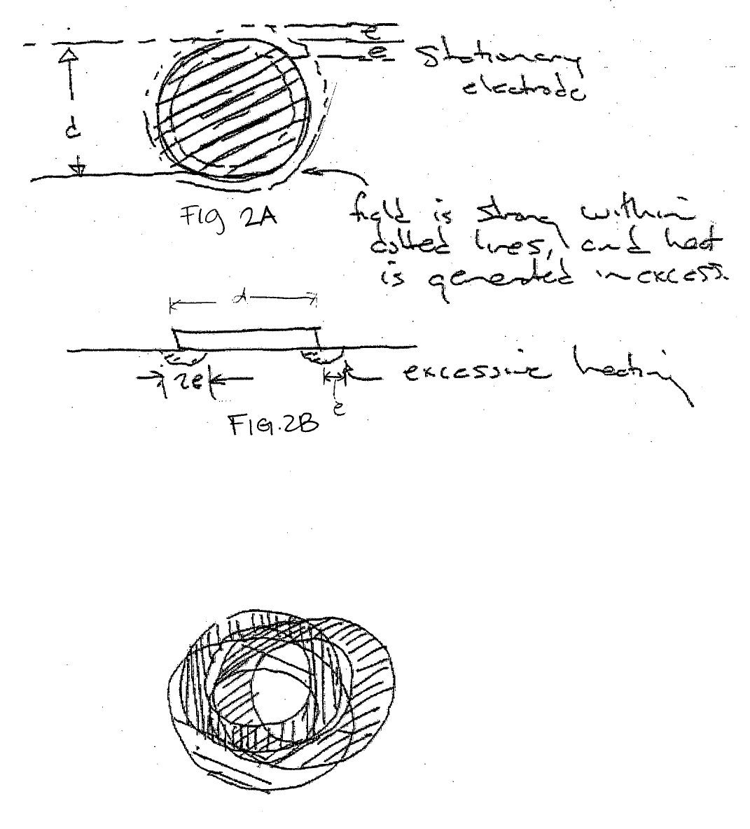

FIGS. 2A and 2B show a plan view and a side section view, respectively, of an electrode contact surface for a circular electrode of diameter d. The lateral extent of edge-effect heating for a stationary electrode edge extends by a distance e in all lateral directions from the electrode edge, creating in the tissue a edge-heated zone in the shape of a ring of width 2e. In general, the distance e is on the order of 1-10 mm.

To control edge effect heating, the amount and direction by which the electrode is moved on the skin is preferably selected so that all edges translate across distances that are larger than the edge heated zone. For a circular electrode, this translates to movement of the electrode within a circular perimeter P of diameter D, where:
D=d+2e

FIG. 2C illustrates the circular perimeter P, with various positions of a circular electrode of diameter d schematically illustrated within the perimeter. Optimal edge effect control will be achieved if the electrode is moved such that its edges reach points along (or outside of) the perimeter. In general, movement of the electrode in a manner that moves all edges of the electrode by at least 1 cm will work well for edge effect control in the disclosed embodiments.

Referring to FIG. 2D, movement of the electrode may be accomplished randomly or by rotating the electrode about an axis of rotation A. Axis A is offset from the center C of the electrode by a distance equal or greater to distance e. A system that automatically rotates the electrode relative to an offset axis is described in detail in connection with FIG. 6.

Cooling

As mentioned, the electrode handpiece 12 preferably includes a cooling element 18 operable to minimize thermal damage to tissue surrounding the subcutaneous tissue that is to be heated during treatment. The cooling element is also useful for offsetting edge heating at the electrode edges.

One form of cooling modality suitable for use with the handpiece 12 is one in which chilled air or cryogen is forced or sprayed onto the tissue surrounding the electrode.

Generally speaking, systems in which the cooling requirements are significant will preferably use forced chilled air or cryogen for tissue cooling and edge effect off-set. These types of cooling are particularly useful in systems in which the electrode is held stationary or only moved very slightly or slowly (e.g. <1 cm/sec). Such systems typically require a significant amount of cooling at the electrode edges. Also, forced cooling systems are preferable if the RF power delivered to the tissue is such (e.g. >25 W/cm²) that it will produce a significant amount of heating over exposure times of minutes or less.

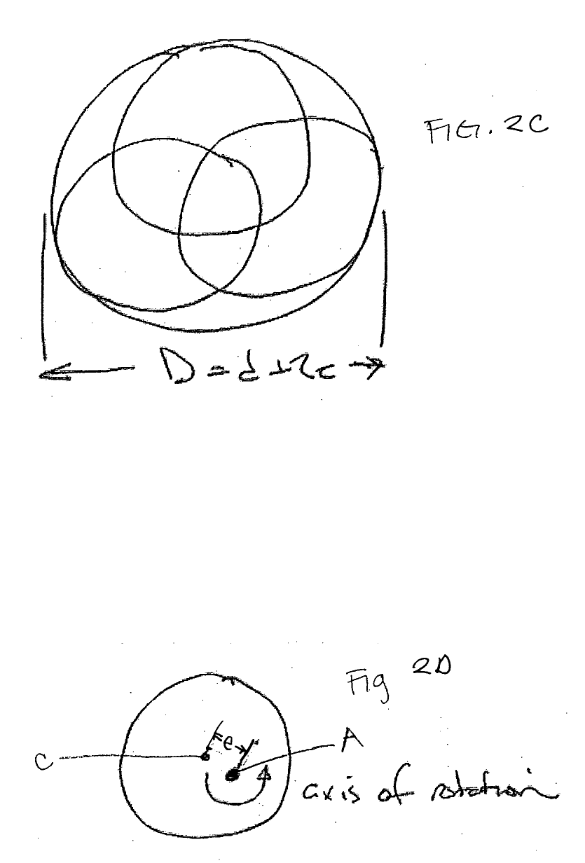

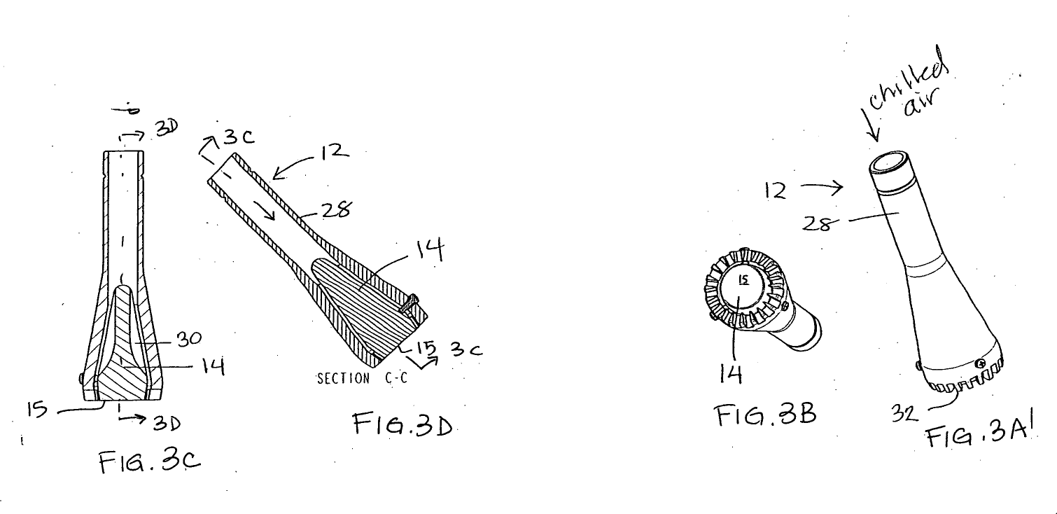

As illustrated in FIGS. 3A-3D, one exemplary handpiece 12 designed for forced chilled air cooling includes a tubular body 28 having an internal lumen that receives chilled air from hose 26 (FIG. 1) as illustrated by arrows. Electrode 14 is disposed within the tubular body 28 and includes a distal surface 15 to be positioned in contact with skin. The electrode 14 includes radial slots 30 or similar features providing channels for passage of chilled air past the electrode and out the distal end of the handpiece 12. Fins 32 in the distal end of the handpiece 12 define approximately radial slots and direct the chilled air in a radially outward direction (or in a direction this is both radial and distal relative to the handpiece), thus allowing the air to impinge on tissue surrounding the handpiece 12 as well as on tissue axially aligned with the handpiece 12.

In other systems, conduction cooling using thermoelectric coolers may be more advantageous than forced air/cryogen cooling. For example, if the electrode is to be moved relatively quickly across the skin surface (e.g. 5 cm/sec or faster), the edge cooling requirements of the system are less than those of stationary electrode systems since the edges of the electrode do not dwell in any particular region of the tissue long enough to produce significant edge heating. In these designs, while forced air/cryogen or other modalities can be used, conduction cooling designs are generally easier to implement are thus are preferable.

The cooling demands are also moderate in embodiments where the ratio of the electrode surface area to that of the treatment area is large (e.g. 3 or higher), making conduction cooling preferable than forced air/cryogen cooling due to its simplicity.

Where the electrode will deliver relatively low intensity RF (e.g. <10 W/cm²), the cooling demands are quite low, and so conduction cooling is appropriate even if the ratio of the electrode surface area to that of the treatment area is low (even <2) and even if the electrode is held near stationary during treatment. Again, forced air/cryogen cooling may be used in this context, but conduction cooling is preferred due to its ease of implementation.

For a very large surface area electrode, force air/cryogen cooling may be particularly difficult to implement because of the large edge perimeter of the electrode. Medical grade forced chilled air/cryo systems, for example, might run out of volume/time they can move, limit the parameters of the RF treatment, or move so much air that it becomes a practical limit to the performance of the treatment itself.

FIGS. 4A, 6, 8 and 9 show embodiments that rely on tissue cooling using an electrode cooled by a thermoelectric cooler, although different types of cooling systems such as those listed elsewhere in this application might instead be used. These embodiments minimize the effects of edge heating through movement of the electrode over the skin surface during the course of energy delivery.

The FIG. 4B applicator 48 includes a relatively small surface area electrode 50. As one example, the electrode might have a tissue contacting surface area of approximately 3 cm². The electrode preferably has a circular contact surface, but any other electrode shape can be used. A preferred cooling device 52 for the applicator 48 is a thermoelectric cooler (“TEC”) positioned in contact with the electrode 50. A heat sink 54 is in thermal contact with the thermoelectric cooler so as to dissipate heat generated by the thermoelectric cooler. The FIG. 10A applicator 48 is mounted on a handpiece (not shown) having a grip or handle oriented to allow a user to glide the electrode 50 over the skin surface as shown in FIG. 4B.

In a preferred method utilizing electrode movement, parameters including the rate of cooling provided by the cooling system, the rate at which the electrode is moved, and the size of the electrode surface in contact with tissue are selected to achieve the desired degree of heating of the target tissue. Experimental results have shown that for an RF treatment period of at least 2 minutes, 100 Watts of RF power from the electrode, with the electrode moving over an area of approximately 20 cm², will produce a surface temperature rise of approximately 10-20 C. For an applicator where cooling is directed to the tissue through the electrode (e.g. using a TEC), the electrode is preferably sized to remove the heat that would cause this surface tissue heating effect, which in this example is approximately 5 W/cm² of skin area. The cooled electrode should also remove the heat that would flow from the native skin to the electrode held at a low temperature (e.g. 10 W/cm² for a target skin surface temperature of 5 C.). Thus, at a target skin temperature of 5 C., the electrode cooling should be able to remove 15 W/cm² (i.e. 10 W/cm² for the heat loading of the skin at 5 C., plus 5 W/cm² of additional RF loading).

To remove 15 W/cm² of heat using a cooled electrode having a skin contact surface of 3 cm², the thermoelectric cooler 52 may be operated to remove (15 W/cm^{2*}3 cm²)=45 W of heat at 5 C.

FIG. 4B illustrates one exemplary pattern for moving the electrode within a target tissue region T.

One example of operating parameters for use of the embodiment of FIG. 4A (with an electrode contact surface of 3 cm²) to treat a tissue area of 20 cm² are as follows:

Power applied to tissue=100 W to achieve 5 W/cm² of tissue heating within the tissue area.

Cooling=45 W as calculated above to maintain a 5 C. skin surface temperature.

Treatment time=3 minutes

Speed of moving electrode=10 cm/sec.

FIG. 5A shows a modification to the design of FIG. 10A in which a standoff 49 is used to electrically isolate the electrode 50a from the thermoelectric cooler 52. The standoff 49 is formed of an electrically isolating material having good thermal conduction properties, such as sapphire. As shown, electrode 50a is mounted within a recess 51 formed in the standoff 49, and may be held in place using screw fasteners 53 passed through holes in the electrode 50a and into corresponding threaded bores formed in the standoff. A layer of thermal expoxy or grease 55 may be positioned between the electrode and the standoff standoff, filling air gaps that could otherwise impede thermal conduction between the standoff and the electrode. The lead 57 for the electrode 50a extends through a channel 59 formed in the standoff 49. The thermoelectric cooler 52 and the heat sink 54 are mounted to the back surface of the standoff 49.

FIG. 5B shows a second embodiment of a design which uses an electrically isolating standoff 49a. In the FIG. 5B embodiment, the standoff is ceramic element coated with a metallic electrode 50b (e.g. gold, copper) on the patient-facing surface of the standoff 49a. The thermoelectric cooler 52 is positioned on the back side of the standoff 49, and the heat sink 54 is positioned on the thermoelectric cooler 52 as shown.

Mounts 61 support the electrode 50b in the handpiece. Electrical contacts 63 are positioned between mounts 61 and the electrode surface, and are attached to leads (not shown) that electrically connect the electrode 50b to the RF system.

The standoff 49a may have a convex surface on the patient facing side to facilitate movement of the electrode across the skin of a patient. The entire applicator tip assembly may be coupled to a spring 65 within the handpiece (not shown). Spring loading the tip assembly helps to keep the electrode in firm contact with the skin despite variations in skin topography.

In a further alternative shown in FIG. 5C, an RF ground plate 47 is positioned to prevent RF energy from coupling into the thermoelectric cooler. In this embodiment, the ground plate 47 is positioned between the thermoelectric cooler 52 and thermally conductive ceramic standoff 49. Heat exchanger 54 is positioned in contact with TEC 52. This embodiment uses a low dielectric constant potting material 45 surrounding the edges of the RF ground 47, standoff 49, and electrode 50 to avoid capacitive coupling between the edges of the electrode 50 back to the RF ground 47 which could result in high fields and uncontrolled energy delivery. In other embodiments, this form of capacitive coupling might instead be avoided by selecting appropriate geometry for the RF ground 47, electrode 50 and associated elements.

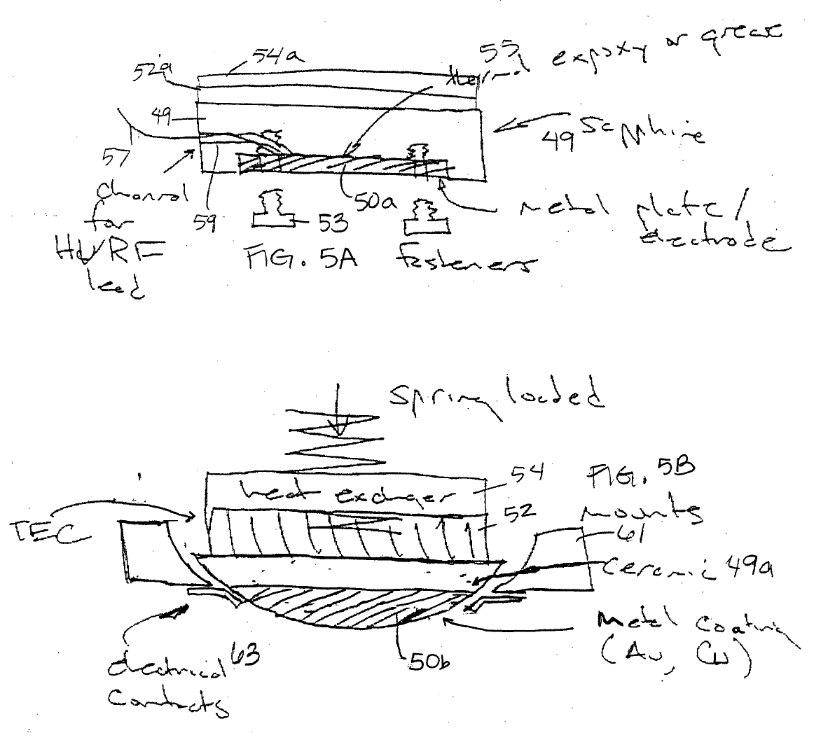

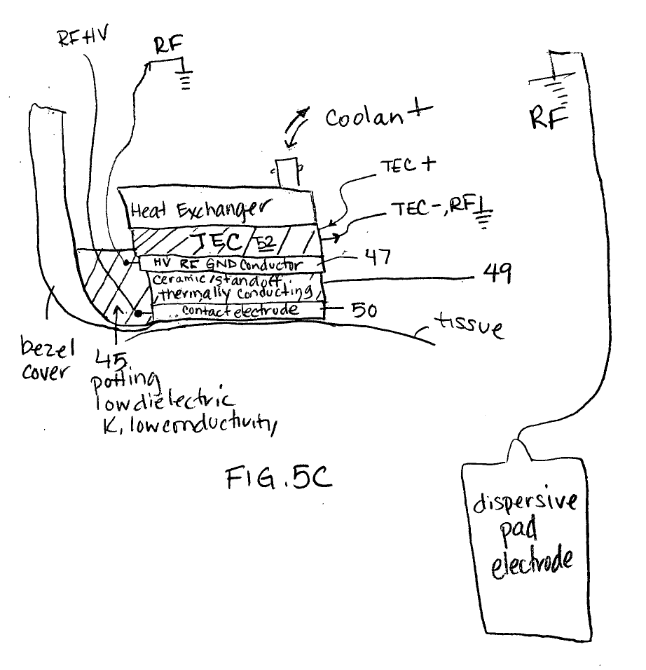

An alternative embodiment of an applicator 55 shown in FIG. 6 utilizes a large surface area electrode 56 (for example, 20 cm²) cooled by a thermoelectric cooler 58, and having a heat sink 60 to dissipate heat from the thermoelectric cooler. In this embodiment, the handpiece includes a motor 62 having a shaft 63 is coupled to the electrode at a position offset from the center of the plane of the electrode. Actuation of the motor rotates the electrode in an off-axis pattern as shown in FIG. 7. The motor and electrode are mounted within a housing (not shown) configured to be held by a user during a treatment cycle, and then repositioned by the user between treatment cycles.

Because a larger electrode surface area is used in the FIG. 6 embodiment, the electrode may be moved more slowly across the area of the skin overlying the target subcutaneous tissue region compared with the movement speed of smaller surface area electrodes. To achieve 5 W/cm² of tissue heating within the target tissue area, the following exemplary set of parameters might be suitable for a tissue area of 30 cm²:

Power applied to tissue=150 W

Cooling=70 W (at 5 C. as above)

Treatment time=3 minutes

Speed of moving electrode=1 cm/sec.

The FIG. 8 embodiment employs an oscillation system for movement of the electrode during energy delivery. For example, the electrode 64, thermoelectric cooler 66 heat sink 68 and a magnet 74 may be supported on the handpiece (not shown) by a mechanical suspension 70. An electromagnet or voice coil 72 is separately positioned on the handpiece. Energization of the electromagnet or voice coil produces lateral vibration of the magnet 74, which causes the electrode to oscillate as indicated by arrows A1, A2. The oscillation of the electrode may be along a single axis as shown, or additional vibration components may be added to cause vibration along multiple axes. In the FIG. 8 embodiment, the skin contacting surface of the electrode 64 has a convex curvature to minimize edge effects during use.

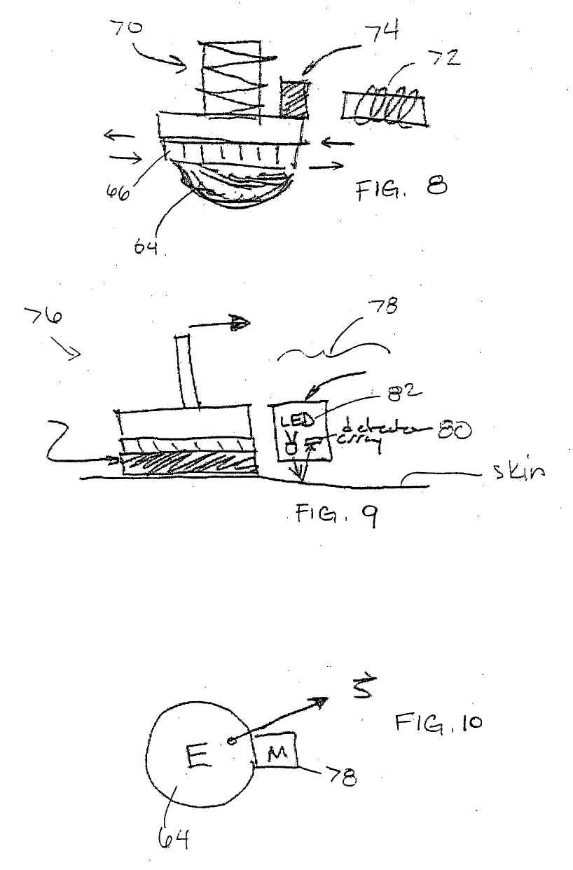

FIG. 9 shows an embodiment in which the applied RF power is varied as a function of the rate at which the electrode is moved across the skin surface. In the FIG. 9 embodiment, applicator 76 includes a detection assembly 78 that generates data representing the speed and direction of motion of the applicator 76 across the skin. The detection assembly 78 may be equipped with features similar to those found on an optical mouse, i.e. an optical detector array 80 and an LED light source 82. Throughout the procedure, light from the LED bounces off the skin onto the detector array which repeatedly sends output to the system for calculating the speed and direction of motion S (FIG. 10) of the applicator. In an alternative embodiment, the detection assembly may instead use an accelerometer or a tracking ball or wheel to determine the direction and speed of movement.

Tracking the movement of the electrode allows the system to modulate the RF power delivered to the tissue based on the determined value of S at a given moment. This feature can help to minimize the chance of tissue injury if the electrode applicator is translated back and forth across an unchanging path, or if movement of the electrode is stopped or significantly slowed during RF delivery. It can also optimize the therapeutic effect of the treatment by ensuring that the therapeutic power delivered to the tissue remains within the therapeutic range despite variations in the speed and direction of electrode movement. The system may additionally include a visual and/or auditory notification sign alertthe user if the electrode is being moved according to movement patterns or speeds that are not optimal for the therapy.

Electrode Designs for Edge Effect Control

Alternate electrode designs for the system 10 will minimize the RF field concentration at the electrode edges so as to minimize heating of the skin beneath the electrode edges. For the purposes of this description, reducing RF field concentration at the electrode edges (relative to more central regions of the electrode) will be referred to as “grading.”

The effects of edge effect heating may be minimized using a variety of electrode types. In the simplest electrode design approach shown schematically in FIG. 11, an ohmic contact is made with a conducting electrode 14b directly contacting the skin S. In this embodiment (and in the FIG. 2A embodiment which also employs ohmic electrode configurations), there is little grading of the RF field at the electrode edge, thus strong edge effects may be experienced. Edge heating of the tissue is controlled using cooling element 18b, which forces chilled air (see arrows A) onto the skin and/or electrode as discussed above in connection with FIGS. 2A, thus preventing significant dermal heating at the electrode edges. Although other cooling systems may alternatively used, in this embodiment aggressive forced chilled air cooling with a high thermal transfer rate is preferable in light of the strong edge effect associated with an ohmic electrode.

As discussed, the electrode 14b and cooling element 18b are preferably arranged to allow for cooling in each of two ways: (i) conductive cooling through direction of the chilled air onto the electrode itself, which in turn conductively cools the skin that is in contact with the electrode; and (ii) convective cooling through impingement of chilled air directly onto the skin near the electrode.

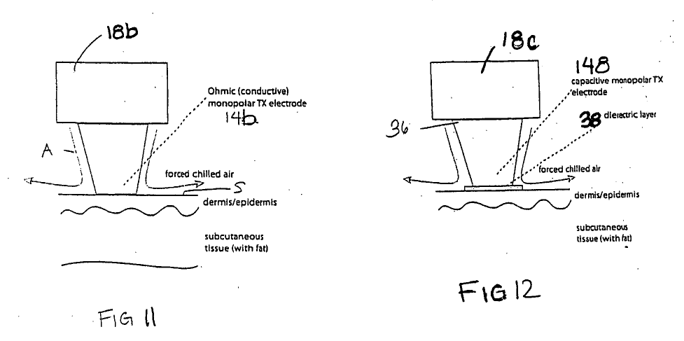

FIG. 12 shows a second embodiment of an electrode 14c which differs from the first embodiment primarily in that the second embodiment uses a capacitive electrode to temper the RF field at the electrode edge. Specifically, in this embodiment the electrode includes a conductive element 36 and a dielectric layer 38 formed of polyimide or other suitable dielectric material. Dielectric layer 38 is positioned such that it will contact the skin S during use, as shown in FIG. 12. The presence of dielectric layer 38 promotes more uniform flow of current through the electrode and into the tissue. The extent of the capacitive effect can be controlled through selection of a dielectric layer having an appropriate thickness and dielectric constant. In preferred embodiments, dielectric layer 38 may have a thickness in the range of 0.0002 to 0.001 inches and a dielectric constant in the range of 3 to 10. Larger dielectric thicknesses and values may be needed where strong edge effects would otherwise occur, and/or the dielectric value and/or thickness may be graded towards the edges of the dielectric layer to offset edge effects. The lateral dimensions of the dielectric layer 38 may exceed those of the conductive element 36 to further offset edge effects.

Where sufficiently thick dielectric layers or large dielectric values are not feasible, a cooling system (e.g. of the type described above) may also be used to offset strong edge heating. As with the first embodiment, a convective cooling modality such as forced air cooling using cooling element 18c, is preferable for off-setting strong edge effect heating.

A third embodiment of an electrode configuration uses a resistive or dissipative electrode in combination with a cooling element.

In one example of a resistive/dissipative electrode shown in FIG. 13, electrode 14d comprises a high voltage RF contact 40d positioned in a cylindrical depression centered on a truncated conical disk 42d of resistive or dissipative material. The disk 42d is constructed of conductive material mixed with an electrically non-conducting, thermally conducting material, such as an elastomer, wax, polymer or polycrystalline insulating material. Examples of material systems useful for this purpose are polyethylene, silicone rubber or RTV doped with carbon black. In this embodiment, skin cooling is achieved through heat conduction through the thermally-conductive/electrically-resistive material to a cooling element 18d employing a cooling modality (e.g. refrigerant, thermoelectric cooler, forced chilled air, etc.). In the FIG. 13 embodiment, the cooling element 18d is located on the side of the electrode opposite the skin.

Electric field grading may be beneficially achieved in the resistive/dissipative electrode configuration. Specifically, the local impedance of the electrode is increased toward the edges of the electrode, thus reducing edge concentration of the applied electric field. Thus, in the FIG. 13 embodiment, the geometry of the resistive/dissipative disk 42d may be graded to achieve a desired increase in impedance from the electrode center towards the electrode edges. FIG. 14 shows one example of a geometrically graded disk 42e which is shaped to include a greater thickness towards the edges than is found in the center. In a preferred geometry, the disk has a progressively increasing thickness from the electrode center region towards the electrode edges, although other shapes having thicker material at the electrode edges may also be used.

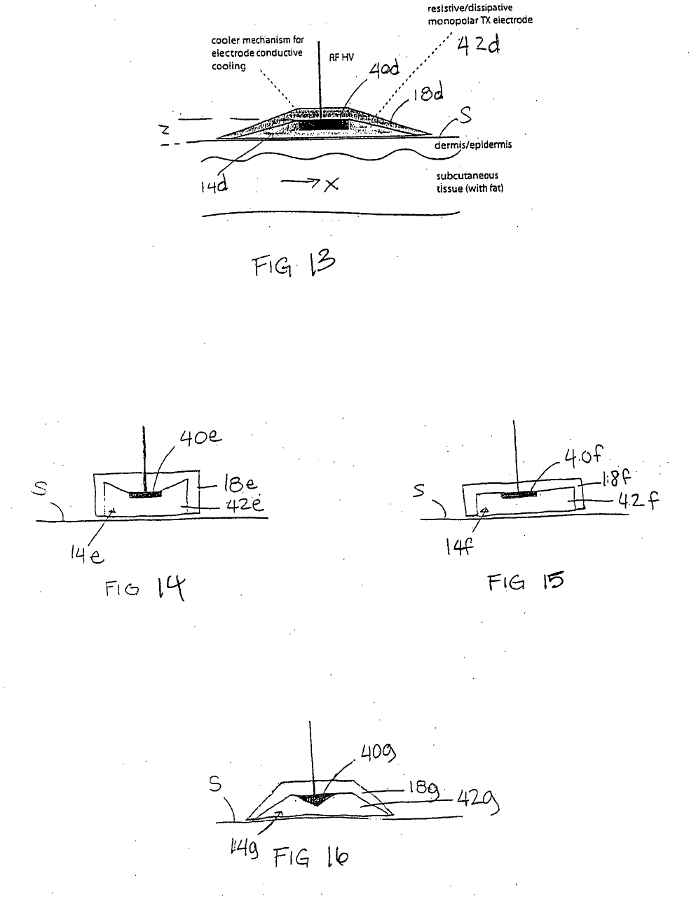

Alternatively, the FIG. 13 embodiment might be modified to achieve grading through variations in the electrical properties of the resistive or dissipative electrode. For example, the resistive/dissipative disk 42f shown in FIG. 15 has generally uniform geometric dimensions, but uses a material system in which the concentration of conductive material is lower (and thus the local impedance is higher) at the edges of the electrode than at more central regions of the electrode. In other embodiments, these FIG. 14 and FIG. 15 approaches to electrode grading may be combined with one another and/or with others disclosed in this application or known to those skilled in the art.

In each of the disclosed approaches, it is desirable to use electrode areas that are large relative to the target depth of heating as described above. Specifically, dimensions and angles should be relatively large compared with layer thicknesses, if edge-heating sensitivity to the local anatomy is to be avoided. A relatively large angle may be loosely defined as one in which the thickness or other dimension associated with an electrode varies by a substantial fraction over lateral distances comparable to skin/fat layer thicknesses. Typically, dermal thickness varies from 0.5 to 2 mm, and subcutaneous layer thicknesses vary from 2-20 mm. Referring to FIG. 13, the vertical dimensions Z of the electrode disk 42d will preferably vary in the lateral dimension x such that dz/dx<10%.

An alternative electrode design shown in FIG. 16 uses electrodes that are relatively insensitive to tissue layer thicknesses and thus will reduce edge heating regardless of the thickness of the skin and fat layers. The FIG. 16 embodiment is similar to the embodiment of FIG. 6, but is modified to use an RF contact 40g having the shape of an inverted cone. In this embodiment, the volume of subcutaneous tissue that is to be heated may be increased by increasing the diameter of the contact 40g.

As with the other embodiments, the FIGS. 13-16 embodiments preferably control dermal heating using cooling elements integrated with the applicator or provided as separate components. Forced air-cooling of the type described above may be used for this purpose. Alternatively, because resistive or dissipative electrode designs of the type described in connection with FIGS. 13 through 16 produce weaker edge heating effects than the ohmic and capacitive electrode designs, these embodiments are suitable candidates for other conductive, convective and/or evaporative cooling methods known in the art.

As one example, conductive cooling may be accomplished using a thermoelectric element as the cooling element 18. Other useful cooling designs include those making conductive use of refrigerants or cryogens, in which, for example a coolant might be circulated inside a chamber positioned within the cooling element. For these embodiments, the electrode is preferably formed of a material having excellent thermal conductivity such as a sintered ceramic or a thermally conductive RTV material such as silicone. Other known cooling systems may also be used, including such as those used in commercially available dermatological laser products.

It should be noted that conductive cooling rates through the dissipative electrode material are low given the low thermal conductivities of the electrode materials. Thus it is preferable to deliver RF power to the tissue slowly in such embodiments to allow the cooling system to keep pace with any skin heating that might occur, keeping in mind however that RF delivery need not be overly slow since the target tissue volume is large and possesses a thermal relaxation time on the order of 10s-100s of seconds.

Operating Parameters

For optimal use of the system, various RF parameters are selected so as to achieve optimal heating at the target depth with minimal collateral tissue heating and edge effects. Generally, the RF frequency should be chosen for maximum heating selectivity in the desired tissue (subcutaneous fat). In a preferred method, frequencies in the range of 0.5-10 MHz are used, although frequencies above and below this range may also be achieve desirable tissue heating.

A slow rate of energy deposition can be used to limit electrode edge heating of the dermis to a level that can be counteracted with surface cooling. Moreover, a relatively slow rate of deposition is suitable for heating of subcutaneous adipose tissue, since the typical volume of the target fat tissue is relatively large compared to the overlying dermis. The very large thermal relaxation time associated with such a large volume (i.e. the time it takes to release ½ the heat it gained by being heated) will be on the order of 10's to 100's of seconds. Thus, an optimal rate of energy deposition can be found for a particular electrode geometry that limits skin edge heating while achieving sufficient subcutaneous fat heating.

Factors to be considered when selecting the appropriate RF dose (e.g. the RF power and the duration of the RF treatment) include the geometry of the skin and subcutaneous tissue in the target region, and the amount of subcutaneous heating necessary to achieve the desired cosmetic result, and the amount of cooling available from the cooling element. The control system 22 monitors the applied RF power, voltage, current and RF exposure time to ensure delivery of the predetermined RF dose. Cooling times (whether before, during, and/or after RF delivery) are also monitored and controlled.

For ohmic electrodes of the type shown in FIGS. 2-3 and 10A-15, typical RF power densities for use of the system are in the range of 2 W/cm²-25 W/cm². Power densities towards the lower end of this range can produce significant subcutaneous heating in approximately 1-2 minutes, whereas power densities in the range of 20 W/cm² or higher can achieve significant heating within approximately 1-10s. In one particular example, an electrode having a contact surface of 2-5 cm² is used, with an applied power of 4-125 W being delivered per therapeutic pulse.

According to a method for using the system 10, skin-contacting electrode 14 is placed against the skin surface overlaying the region of fat that is to be treated. The user depresses footswitch 23, causing the electrode to conduct RF current into the tissue for a desired amount of time. As discussed above, depending on the system used, the handpiece 12 may be held in place in one position on the skin, or moved manually or automatically over a target treatment region during RF delivery.

If conduction cooling is used, the cooling element cools the electrode, which in turn cools the skin in contact with the electrode. If forced air/cryo cooling is used, the epidermal cooling system 20 directs chilled air into the handpiece, thus forcing the air onto the skin and into contact with the electrode. The forced chilled air convectively cools the electrode and the skin, and also conductively cools the skin using the cooled copper electrode 14. The cooling system 20 and/or thermoelectric cooler may be operated to cool the tissue and/or the electrode 14 before, during, and/or after RF delivery to prevent thermal damage to the superficial skin layers. Pre-cooling (i.e. prior to delivery of RF energy) of the dermis overlaying the target region of subcutaneous fat can be useful for lowering the temperature of the dermis by an amount sufficient to prevent the rise in dermal temperature during RF delivery from exceeding that which would cause thermal injury to the dermis. In other words, when the electrode is energized, the pre-cooled dermal tissue is protected from thermal damage that might otherwise result from RF delivery. Pre-cooling is ideally performed for a period of time calculated to cool a pre-determined thickness of the dermis below a predetermined temperature. Activation of the cooling system for a period of time following RF delivery can beneficially prevent the RF-heated subcutaneous fat layers from conducting heat to the dermis in amounts sufficient to cause thermal damage to the dermis. After a predetermined RF delivery time, the electrode 14 is repositioned to treat one or more additional regions.

In response to application of RF energy, cosmetic changes to the subcutaneous fat, adipose tissue, or cellulite proceed through means of a controlled or dosed thermal injury or insult to a spatially localized region of subcutaneous tissue. The injured tissue may undergo direct thermolipolysis as an immediate reaction. Alternatively, the treatment may produce sufficient injury to cells such that over time the tissue is partially resorbed as part of a wound response, or as the result of cellular responses triggered by biochemical signaling of the type that accompanies the stress or injury reaction of other cells or tissues, or as the result of neural signaling mediated by thermal stress (e.g. sympathetic nerve control of lipolysis as postulated by S. Klaus, Ph.D, Brown Adipose Tissue: Thermogenic Function and Its Physiological Regulation, Adipose Tissue, Medical Intelligence Unit 27, page 76.). In some treatments, the non-adipose tissue structures in the subcutaneous might contribute to improved cosmetic appearance by several mechanisms. For example, it is believed that strong preferential heating of fibrous septae can result from exposure to RF energy. M. T. Abraham et al, Current Concepts in Nonablative Radiofrequency Rejuvenation of the Lower Face and Neck, Facial Plastic Surgery, July 2005. Destruction, shrinkage, denaturation or subsequent fibrosis and scarring of these structures can have significant effects on the appearance of the treated region, including but not limited to diminution of the appearance of cellulite.

While various embodiments of the present invention have been described above, it should be understood that they have been presented by way of example, and not limitation. It will be apparent to persons skilled in the relevant art that various changes in form and detail may be made therein without departing from the spirit and scope of the invention. This is especially true in light of technology and terms within the relevant art(s) that may be later developed. Additionally, it is contemplated that the features of the various disclosed embodiments may be combined in various ways to produce numerous additional embodiments. Thus, the present invention should not be limited by any of the above-described exemplary embodiments, but should be defined only in accordance with the following claims and their equivalents.

Any and all patents, patent applications and printed publications referred to above are incorporated by reference.

We claim:

1. A method for treating subdermal fat tissue, the method comprising:
delivering energy to an RF electrode in contact with skin overlying a target sub-dermal fat region while continuously moving the RF electrode along the surface of the skin.

2. The method of claim 1, wherein the delivering step reduces, removes, shapes and/or sculpts the sub-dermal fat tissue.

3. The method of claim 1, wherein the sub-dermal fat tissue is adipose tissue and/or cellulite.

4. The method of claim 1, wherein the step of delivering energy heats the sub-dermal fat tissue.

5. The method of claim 1, wherein the method further includes cooling the skin overlaying the sub-dermal fat region.

6. The method of claim 5, wherein cooling the skin includes impinging chilled air onto the skin.

7. The method of claim 5, wherein cooling the skin includes cooling the electrode, and causing the cooled electrode to cool the skin.

8. The method of claim 5, wherein cooling the skin step includes impinging chilled air onto the skin and the electrode.

9. The method of claim 1, wherein delivering energy to an RF electrode heats fibrous septae in the subcutaneous fat tissue.

10. A method for treating sub-dermal fat tissue, the method comprising:
delivering energy to the skin using an RF electrode in contact with skin overlying the tissue; and
impinging chilled air onto the RF electrode and onto skin adjacent to the electrode.

11. The method of claim 10, wherein delivering energy reduces, removes, shapes and/or sculpts the sub-dermal fat tissue.

12. The method of claim 10, wherein the sub-dermal fat tissue is adipose tissue and/or cellulite.

13. The method of claim 10, wherein delivering energy heats the sub-dermal fat tissue.

14. The method of claim 10, wherein delivering energy heats fibrous septae in the subcutaneous fat tissue.

15. The method according to claim 1, wherein the method further includes determining the speed and/or direction of movement of the electrode.

16. The method according to claim 15, further including modulating applied RF power based on the determined speed and/or direction of movement.

17. The method according to claim 15, further including terminating power delivery if the determined speed falls below a predetermined level.

18. The method according to claim 15, further including terminating power delivery if a rate of change of the determined direction of movement is below a predetermined level.

19. The method according to claim 1, wherein a direction and rate of movement of the RF electrode is selected to minimize edge heating effects.

20. The method according to claim 19, wherein the RF electrode has an edge and wherein RF electrode is moved over the surface of the skin by an amount of at least 0.5 cm in a lateral direction.

21. The method according to claim 15, wherein a light source and optical detector are moveable with the RF electrode, and wherein determining the speed and/or direction of movement includes reflecting light off the skin using the light source, and detecting the reflected light using the optical detector.

22. The method according to claim 15, wherein an accelerometer is moveable with the RF electrode, and wherein determining the speed and/or direction of movement is performed using feedback from the accelerometer.

23. The method according to claim 15, wherein a tracking ball is moveable with the RF electrode, and wherein determining the speed and/or direction of movement is performed using feedback from the tracking ball.

24. The method according to claim 1, including automatically moving the RF electrode over the surface of the skin.

25. The method according to claim 24, wherein automatically moving the RF electrode includes rotating the RF electrode relative to an axis laterally off-set from a rotational center point of the RF electrode.

26. The method according to claim 25, wherein automatically moving the RF electrode includes oscillating the electrode across the surface of the skin.

27. A system for treating tissue, the system including;
an RF power supply;
a handpiece;
an electrode carried by the handpiece and electrically coupled to the RF power supply; and
a motion detector coupled to the handpiece, the motion detector positioned to detect speed and/or direction of movement of the electrode across the surface of skin tissue.

28. The system according to claim 27, wherein the motion detector comprises an optical motion detector comprising a light source and an optical detector.

29. The system according to claim 27, wherein the motion detector comprises an accelerometer.

30. The system according to claim 27, wherein the motion detector comprises a trackball.

31. The system according to claim 27, wherein the RF power supply include a controller responsive to feedback from the motion detector to modulate RF power based on the determined speed and/or direction of movement.

32. The system according to claim 27, wherein the RF power supply includes a controller responsive to feedback from the motion detector, the controller operable to terminate power delivery to the electrode if the determined speed falls below a predetermined level.

33. The system according to claim 27, wherein the RF power supply includes a controller responsive to feedback from the motion detector, the controller operable to terminate power delivery to the electrode if a rate of change of the determined direction of movement is below a predetermined level.

34. The system according to claim 33, wherein the handpiece has at least one channel, and wherein the system further includes a source of cooling fluid fluidly coupled to the channel; the at least one channel positioned to permit cooling fluid from the source to be impinged onto the electrode and out of a distal portion of the handpiece.

35. The system according to claim 34, wherein the channel is positioned to permit cooling fluid to exit the channel in an annular pattern surrounding the electrode.

36. The system according to claim 34, wherein the channel is positioned to permit cooling fluid to exit the handpiece in a radial direction.

37. The system according to claim 34, wherein the electrode includes a lateral surface and a plurality of longitudinal slots in the lateral surface.

38. The system according to claim 34, wherein the handle includes a plurality of radial slots at its distal end.

39. The system according to claim 27, wherein the electrode is selected from the group consisting of ohmic electrodes, capacitive electrodes, and resistive electrodes.

40. The system according to claim 27, wherein the electrode comprises a copper electrode.

41. The system according to claim 40, further including a cooler positioned to cool the copper electrode.

42. The system according to claim 41, wherein the cooler is a thermoelectric cooler.
